(19) United States
(12) Patent Application Publication
(10) Pub. No.: US 2010/0056636 A1
(43) Pub. Date: Mar. 4, 2010

(54) N-Substituted-P-Menthane-3-Carboxamide and Uses Thereof

(76) Inventors: Stefan Michael Furrer, Cincinnati, OH (US); Sander Tondeur, Loosdrecht (NL); Chris Winkel, Bussum (NL); Harry Renes, Lelystad (NL); Adri De klerk, Naarden (NL)

Correspondence Address: CURATOLO SIDOTI CO., LPA, 24500 CENTER RIDGE ROAD, SUITE 280, CLEVELAND, OH, 44145, US

(21) Appl. No.: 12/519,801

(22) PCT Filed: Nov. 30, 2007
(86) PCT No.: PCT/NL07/50609

§ 371 Date: Aug. 28, 2009

Related U.S. Application Data
(60) Provisional application No. 60/982,464, filed on Oct. 25, 2007.

(30) Foreign Application Priority Data
Dec. 20, 2006 (EP) 06126622.7

(51) Int. Cl. A61K 31/166; C07C 233/57; A61P 25/00

(52) U.S. Cl. 514/617; 564/184

(57) ABSTRACT

The present invention relates to substances and compositions having a physiological cooling effect on the skin and the mucosa of the body, especially of the oral cavity, throat and nose. More in particular, the present invention relates to N-substituted-p-menthane-3-carboxamides represented by the following formula (I):

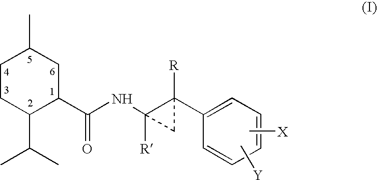

and esters thereof. These substances are capable of imparting and/or enhancing a physiological cooling effect in a product in which they are incorporated, much more effectively than the heretofore known N-substituted-p-menthane-3-carboxamides.

### FIELD OF THE INVENTION

The present invention relates to substances and compositions having a physiological cooling effect on the skin and the mucosa of the body, especially of the oral cavity, throat and nose. More in particular, the present invention relates to N-substituted-p-menthane-3-carboxamides and especially to flavour and/or fragrance compositions comprising such a substance, the use thereof for imparting or enhancing a physiological cooling effect in a consumer product, and to consumer products comprising such a substance.

### BACKGROUND OF THE INVENTION

In the field of flavours and fragrances for consumer products such as foodstuffs, beverages, tobacco products and personal care products there has always been great interest in compounds having a physiological cooling activity on the nervous system of the body, especially of the skin and the mucosa of the oral cavity, similar to that obtained with menthol.

Such compounds may be added to ingestible preparations, to tobacco products, and/or to products applied to the skin, for the purpose of stimulating the cold receptors of the nervous system in the surface tissues of the mucosa of the oral cavity or the skin, thereby creating a sensation of coolness and/or freshness in the mouth or on the skin.

Menthol has been used extensively for this purpose, and mainly as a fortifier for peppermint flavours (see “Perfume and Flavour Chemicals, Volume II, by Steffen Arctander, published 1969, Item No. 1840) but also in trace amounts in imitation butter, caramel, fruit complexes and licorice flavourings. Menthol is well known for its physiological cooling effect on the skin and mucous membranes of the mouth and has been extensively used as a flavouring agent (menthol being a major constituent of oil of peppermint) in foodstuffs, beverages, dentifrices, mouthwashes, etc. and as a component in a wide range of toiletries, liniments and lotions for topical application. Menthol is also a well known tobacco additive for producing a “cool” sensation in the mouth when smoking.

It is well established that the “cooling” effect of menthol is a physiological effect due to the direct action of menthol on the nerve endings of the human body responsible for the detection of hot or cold and is not due to latent heat of evaporation. It is believed that the menthol acts as a direct stimulus on the cold receptors at the nerve endings which in turn stimulate the central nervous system.

Although menthol is well established as a physiological coolant, its use in some compositions is impaired by its strong minty odour and its relative volatility.

A few other compounds similar to menthol and possessing physiological cooling activity have also been used in trace amounts in flavourings to give lift and freshness, e.g. menthone (Perfume and Flavour Chemicals, Item 1843) menthyl acetate and propionate (Perfume and Flavour Chemicals, Items 1845 and 1852), and 3,3,5-trimethylcyclohexanol (Perfume and Flavour Chemicals. Item 2998). Japanese Pat. No. 39-19627 reports that 3-hydroxymethyl p-menthane (menthyl carbinol) has a flavour closely resembling that of 1-menthol and suggests its use as a flavourant in confectionery, chewing gum and tobacco. In Swiss Pat. No. 484,032 certain saccharide esters of menthol are proposed as additives for tobacco. In French Pat. no. 1,572,332 N,N-Dimethyl 2-ethylbutanamide is reported as having a minty odour and refreshing effect, and the minty odour of N,N-diethyl 2,2-dimethylpropanamide is referred to. A similar effect is reported for N,N-diethyl 2-ethylbutanamide in Berichte 39, 1223, (1906). A minty odour has also been reported for 2,4,6-trimethylheptan-4-ol and 2,4,6-trimethyl hept-2-en-4-ol in Parfums-Cosmetiques-Savons, May 1956, pp. 17-20. The cooling effect of menthol and other related terpene alcohols and their derivatives has also been studied and reported in Koryo, 95, (1970), pp. 39-43. 2,3-p-Menthane diol has also been reported as having a sharp cooling taste (Beilstein, Handbuch der Organischen Chemie. 4th Ed. (1923) Vol. 6, p. 744.).

In U.S. Pat. No. 4,136,163 (Wilkinson Sword) N-substituted-p-menthane-3-carboxamides are disclosed having the property of stimulating the cold receptors of the nervous system of the human body to produce a cold sensation. These compounds have little or no odour, are of relatively low volatility and are substantially non-toxic. According to U.S. Pat. No. 4,136,163 the cooling sensation created by these N-substituted carboxamides on the skin, and mucous membranes, for example in the mouth, varies both in intensity and longevity from compound to compound. In U.S. Pat. No. 4,136,163 one alkylphenyl substituted p-menthane-3-carboxamide is disclosed.

Eversince the aforementioned work by Wilkinson Sword was published there has been great interest in finding and developing the most potent N-substituted-p-menthane-3-carboxamides, i.e. having the most intense and/or long lasting effects. Of the original Wilkinson Sword N-substituted-p-menthane-3-carboxamides, three have been successfully commercialized: WS-3 (N-Ethyl-p-menthane-3-carboxamide), WS-5 (Ethyl 3-(p-menthane-3-carboxamido)acetate) and WS-14 (N-tert-butyl-3-p-menthane carboxamide). WS-5 is known to be significantly cooler than WS-3. In US Patent Application No. 20050222256 it has been reported that, for “highly purified (1R,3R,4S)-WS-5”, the perceived cooling is about 2.5-3.0 times stronger than WS-3. See also Mark Erman, Progress in Physiological Cooling Agents. Perfumer & Flavorist, Vol. 29. No. 8, pp. 34-50 (2004).

Further series of N-substituted-p-menthane-3-carboxamides have been the subject of more recent patents and patent applications. For example, in Japanese Patent No. 2004059474, a new series of cooling compounds based on alkyloxy amides of p-menthane has been disclosed.

WO 2005/049553 and WO 2006/049553 both disclose N-substituted-p-menthane-3-carboxamides wherein the carboxamide-substituent is a substituted aromatic which is connected to said carboxamide moiety directly, i.e. through a covalent bond, or through an unsubstituted methylene group.

A comprehensive review of cooling compounds known in the field of flavours and fragrances is provided by Leffingwell et al. (http://leffingwell.com; “Cooler than menthol”, updated Apr. 5, 2006).

As will be clear from the above, there is still a desire for finding new cold receptor stimulants, especially cold receptor stimulants that are even more potent than the ones presently available. The present invention thus seeks to provide such a cold receptor stimulant.

### SUMMARY OF THE INVENTION

The present inventors have surprisingly found that substances represented by the following formula (I):

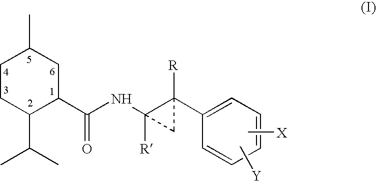

and esters thereof are capable of imparting and/or enhancing a physiological cooling effect in a product in which they are incorporated, much more effectively than the heretofore known N-substituted-p-menthane-3-carboxamides, more in particular, this substance was found to be significantly more potent, especially stronger and/or longer-lasting, than any of the prior art N-substituted-p-menthane-3-carboxamides.

Hence, it has been found that a very intense and/or very longer lasting physiological cooling effect can be imparted in a consumer product selected from foodstuffs, beverages, oral care products, cosmetic products, personal care products and tobacco products, by the incorporation of an effective amount of one or more of the aforementioned cold receptor stimulants represented by said formula (I) and/or edible esters thereof, said effective amount being unexpectedly low. As will be described hereafter the strength of the present cold receptor stimulant is considerably higher than that of WS-3, approximately a factor 10 higher, and that of WS-5, which so far has been assumed to constitute the most potent cold receptor stimulant of the N-substituted-p-menthane-3-carboxamide family.

Substances structurally resembling the cold receptor stimulants of the present invention have previously been disclosed in WO 2005/020897 as an example of an agonist of Trp-p8, which is a cation channel protein, preferentially expressed in the prostate and found over-expressed in a range of cancers, including prostate breast, lung and colon cancer. According to WO 2005/020897, modulation of Trp-p8 activity by activation with an agonist can be valuable as a therapeutic to manipulate the Trp-p8 expressing cells in a specific manner, typically inhibiting growth thereof and/or inducing apoptosis and/or necrosis thereof. Oral or topical administration of said substance is not disclosed, the preferred route of administration is parenteral. Nothing is disclosed in WO 2005/020897 teaching or suggesting the skilled person that the substances of the present invention impart and/or enhance physiological cooling effects on the skin and/or mucosa.

Hence the present invention provides for the first time the substances represented by the aforementioned formula (I) and their use as a cold receptor stimulant for imparting and/or enhancing a physiological cooling effect in a consumer product, especially in a consumer product selected from foodstuffs, beverages, oral care products, cosmetic products, personal care products and tobacco products. Accordingly, the invention also provides cooling compositions comprising one or more of the present cold receptor stimulants as well as one or more flavour imparting substances and/or fragrance materials, which can suitably be applied for imparting and/or enhancing said cooling effects as well as methods of imparting and/or enhancing a physiological cooling effect therein, especially coolness and/or freshness.

### DETAILED DESCRIPTION OF THE INVENTION

Accordingly, a first aspect of the invention concerns a cold receptor stimulant selected from the group of substances of the following formula (I):

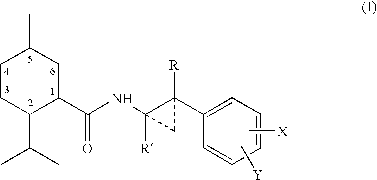

wherein, R represents hydrogen, hydroxyl, oxo, lower alkyl or lower alkoxyl;
R′ represents hydrogen or lower alkyl;
X and Y independently represent hydrogen, hydroxyl, lower alkyl and lower alkoxyl;
and wherein the dashed lines represent an optional additional bond or an optional C₁-C₂ alkylene moiety; and esters thereof and at least 0.1 wt % of one or more flavour imparting substances and/or one or more fragrance imparting substances.

A preferred embodiment of this invention concerns cold receptor stimulants as defined above selected from the group of substances of the following formula (II):

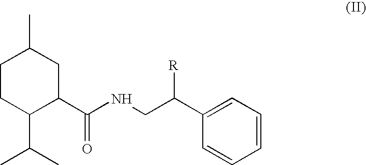

wherein, R represents hydrogen, hydroxyl lower alkyl or lower alkoxyl; and esters thereof.

The present inventors have found that the above-mentioned cold receptor stimulants are very useful ingredients for application in foodstuffs, beverages, personal care products and tobacco products, said cold receptor stimulants being capable of imparting and/or enhancing a physiological cooling effect in a product to which it is added. Throughout this document the term “physiological cooling effect” is used to describe the specific sensorial impact that is perceived via the skin and mucosa, especially in the oral and nasal cavities as well as the throat.

As used herein the term ‘esters thereof’ encompasses any derivative of the present cold receptor stimulant and an acid formed by reaction of said acid with the hydroxyl group present in said substance. Preferably said esters are edible, typically they are not significantly more toxic than the cold receptor stimulant represented by formula (I). Typically said acid is an organic acid such as a substituted or non-substituted, linear or branched C₁-C₆ carboxylic acid, more preferably a C₁-C₄ carboxylic acid, most preferably a C₁-C₃ carboxylic acid, or alternatively an inorganic acid such as phosphoric acid. Such esters will at least to a certain degree, possess some of the sensory characteristics of the corresponding substance according to formula (I) that is not esterified. According to a preferred embodiment of the invention, the cold receptor stimulant is selected from the group consisting of substances of formula (I).

The term “comprising” whenever used in this document is intended to indicate the presence of stated features, integers, steps, components, but not to preclude the presence or addition of one or more other features, integers, steps, components or groups thereof.

The term “lower” as used herein in connection to the term “alkoxyl” and “alkyl”, means that the moiety concerned comprises a carbon chain portion of not more than six carbon atoms, preferably of not more than four carbon atoms, most preferably of not more than two carbon atoms. In a preferred embodiment the lower alkyl is a branched or unbranched saturated C₁-C₆ alkyl, preferably C₁-C₄ alkyl, most preferably C₁-C₂ alkyl. In another preferred embodiment the lower alkoxyl is a branched or unbranched saturated C₁-C₆ alkoxyl, preferably C₁-C₄ alkoxyl, most preferably C₁-C₂ alkoxyl.

In a particularly preferred embodiment of the present invention, a cold receptor stimulant as defined herein before is selected wherein R represents hydrogen, hydroxyl, methyl, ethyl, methoxy, ethoxy, isopropoxy, and propoxy; R′ represents hydrogen, methyl or ethyl; X and Y independently represent hydrogen, methyl, ethyl, propyl, isopropyl, hydroxyl, methoxy, ethoxy, isopropoxy or propoxy and the dashed lines represent an optional double bond or an optional methylene moiety.

In a particularly preferred embodiment of the invention, R in formula (I) and (II) represents hydrogen, hydroxyl, methoxy or methyl most preferably hydrogen or hydroxyl.

It was found that the cold receptor stimulant according to formula (I) or (II) wherein R represents hydrogen is even slightly stronger than that wherein R represents hydroxyl. Hence, in a most preferred embodiment of the invention, R in formula (I) or (II) represents hydrogen.

In another particularly preferred embodiment of the invention R′ represents hydrogen, hydroxyl, methoxy or methyl most preferably hydrogen.

In a particularly preferred embodiment of the invention, X and Y in formula (I) represent hydrogen, hydroxyl, methyl, ethyl, methoxy or ethoxy, more preferably hydrogen, methyl, methoxy or ethoxy. It is furthermore particularly preferred that X is in the meta or para position. It is furthermore particularly preferred that Y is in the ortho or meta position. Most preferably X and Y are chosen such that aromatic radical in formula (I) represents one of the following: an unsubstituted phenyl radical; a 2-ethoxyphenyl radical and a 3,4-dimethylphenyl radical.

As noted herein before, the dashed lines indicate the optional presence of an additional covalent bond or of a C₁-C₂ alkylene moiety, preferably a methylene moiety, said moiety forming a cyclic structure together with the carbon atoms to which it is attached. Without wishing to be bound by any particular theory, the present inventors believe that the presence of the additional bond or the alkylene moiety limit the rotational degrees of freedom are restricted as compared to a corresponding structure without the additional bond or alkylene moiety, thereby increasing the cold receptor stimulating efficacy of the structure. Hence, in one particularly preferred embodiment of the invention, the present cold receptor stimulants comprise an additional covalent bond or a C₁-C₂ alkylene moiety at the position indicated by the dashed lines in formula (I).

Particularly preferred examples of cold receptor stimulants according to the invention include 2-isopropyl-5-methyl-cyclohexanecarboxylic acid (2-hydroxy-2-phenylethyl)-amide, 2-isopropyl-5-methyl-cyclohexanecarboxylic acid phenylethyl-amide, N-(2-ethoxyphenethyl)-2-isopropyl-5-methylcyclohexanecarboxamide. 2-isopropyl-5-methyl-N-((1S,2S)-2-phenylcyclopropyl)cyclohexanecarbox-amide, N-(3,4-dimethylphenethyl)-2-isopropyl-5-methylcyclohexanecarboxamide and esters thereof.

The specific stereochemical arrangement of the asymmetrical carbon atom of the menthane moiety of the substances represented by formula (I) and (II) may typically affect their relative potency as a cold receptor stimulant. In a particularly preferred embodiment of the invention carbon atom no. 1 of the present cold receptor stimulant is in the R-configuration, carbon atom no. 2 is in the S-configuration and carbon atom no. 5 is in the R-configuration.

A second aspect of the present invention concerns compositions comprising a cold receptor stimulant represented by the above formula (I) or (II), wherein R, R′, X, Y and the dashed lines have the same meaning as defined above, as well as at least 0.1 wt % of one or more flavour imparting substances and/or one or more fragrance imparting substances.

These compositions typically constitute flavour compositions or fragrance compositions. Such compositions are not regarded as consumer products, i.e. are not suitable for consumption and/or use by a consumer as such, but are suited for application in such consumer products, in order to impart or enhance therein the physiological cooling effects, as will be understood by the person skilled in the art.

Preferably the present flavour or fragrance compositions comprise the cold receptor stimulant of the invention in an amount of at least 2 ppm, calculated on dry solids weight, preferably in an amount within the range of 5-100,000 ppm, more preferably within the range of 10-50,000 ppm, most preferably within the range of 50-10,000 ppm.

The expression “ppm” as used herein refers to amounts expressed in parts per million, whereby 1 ppm corresponds to 1 mg/kg, in accordance with its usual meaning in the art of flavours and fragrances.

As used herein the term “flavour imparting substance” encompasses any food grade substance that is capable of imparting a detectable sensory impact that is perceived via the mouth, especially the tongue, and the olfactory epithelium in the nasal cavity, typically at concentrations below 1 wt. %, more preferably below 0.1 wt. %. Suitable examples of flavour imparting substances include alcohols, aldehydes, ketones, esters, ethers, acetates, nitriles, terpene hydrocarbons, nitrogenous or sulphurous heterocyclic compounds. Typically, one or more flavour imparting substances may be comprised in essential oils. Flavour imparting substances in accordance with the invention can be of natural or synthetic origin. Many of these are listed in reference texts such as the book by S. Arctander, Perfume and Flavor Chemicals. 1969, Montclair, N.J., USA, or its more recent versions, or in other works of a similar nature, as well as in the abundant patent literature in the field of flavours. It will be clear to the skilled person that the type of flavour imparting substance added would entirely depend on the type of consumer product to which the composition is added.

In a particularly preferred embodiment of the invention compositions as defined herein before are provided, comprising one or more flavouring substance containing oils, preferably selected from peppermint oil, spearmint oil, other mint oils, clove oil, oil of wintergreen, oil of eucalyptus, oil of anise, and oil of cinnamon.

In a preferred embodiment the composition according to the invention comprises one or more flavour imparting substances in an amount of at least 0.1 wt %, calculated on dry solids weight, preferably in an amount within the range of 0.5-99.wt %, more preferably within the range of 1-50 wt %, most preferably within the range of 2-25 wt %.

As used herein the term “fragrance imparting substance” is used to indicate any odouriferous substance that is used to impart an overall pleasant odor profile to a composition, particularly a cosmetic composition or personal care product. A wide variety of substances are useful as fragrance imparting substances, including e.g. aldehydes, ketones and esters, which may be synthetic or may be derived from naturally occurring plant or animal sources. Naturally occurring plant and animal oils and exudates comprising complex mixtures of various fragrance imparting substances are known as well for use as fragrance materials. Fragrance imparting substances that may suitably be used in accordance with this invention include pro-fragrances such as acetal pro-fragrances, ketal pro-fragrances, ester pro-fragrances, hydrolysable inorganic-organic pro-fragrances and mixtures thereof. Lists of suitable fragrance imparting substances as well as materials comprising them can be found in Journals used by those in the art such as “Perfume and Flavourist” or “Journal of Essential Oil Research”.

Preferably, one or more fragrance imparting substances are comprised in the composition of the invention at a level within the range of 0.5-99 wt. %, calculated on dry solids weight. Preferably the one or more fragrance imparting substances are present at a level within the range of 1-50 wt %, most preferably within the range of 2-25 wt %, by dry weight of the composition.

Typically, in the present compositions the cold receptor stimulant and the one or more flavour imparting substances or the one or more fragrance imparting substances as defined herein before are employed in a weight ratio within the range of 0.001-10, preferably within the range of 0.01-1, most preferably within the range of 0.05-0.5.

In a particularly preferred embodiment of the invention the present cooling composition comprises a further cold receptor stimulant. Suitable examples thereof include menthol, WS-3, WS-23, WS-5, menthyl-lactate, menthoxypropane-1,2-diol, 3-(10-menthoxy)-2-menthylpropane-1,2-diol, (−)-isopulegol, menthyl pyrrolidone carboxylate, cubelol and N,N-dimethyl menthyl succinamide.

The composition according to the present invention may suitably be prepared in the form of a liquid, a paste or a powder, further comprising a carrier material, such as maltodextrin, modified starch, gum Arabic, ethanol or propylene glycol.

In case the composition is a flavour composition it is particularly preferred that the composition is a free flowing powder. Typically such flavour compositions comprise a food grade carrier material. i.e. a carrier material which is non-toxic and does not significantly affect the organoleptic properties of the combination of the cold receptor stimulant and the one or more flavour imparting substances.

In another preferred embodiment a composition is provided comprising a cold receptor stimulant and one or more flavour imparting substances as defined herein before, wherein one or more of said components are encapsulated in a matrix, typically a carbohydrate matrix, which is suitable for retaining volatile components and forms a barrier to oxygen and/or moisture. In a particularly preferred embodiment of the invention one or more of said components are encapsulated in a controlled release matrix, e.g. for chewing gum applications, such as the matrices described in WO2005/084458.

In another aspect of the invention, a consumer product is provided selected from foodstuffs, beverages, oral care products, cosmetic products, personal care products and tobacco products, comprising a cold receptor stimulant as defined herein before, in an amount effective to impart or enhance therein a physiological cooling effect.

According to a particularly preferred embodiment of the invention, a consumer product is provided as defined herein before, wherein said amount is an amount ranging from 0.0001 to 500 ppm, based on the total weight of said product, preferably an amount ranging from 0.0005 to 50, more preferably ranging from 0.001 to 10 ppm, most preferably ranging from 0.002 to 5 ppm.

According to another particularly preferred embodiment of the invention said consumer product is a foodstuff selected from the group of confectioneries, including hard and soft candies, chewing gum, edible films, lozenges and pastilles, desserts and ice cream; a beverage selected from the group of soft drinks, alcoholic beverages and dairy drinks; an oral care product selected from the group of toothpastes, mouthwashes, dental floss, anti-plaque and anti-gingivitis compositions; a personal care product selected from the group of deodorants, shampoos, skin sanitizing compositions, lotions and shaving products; or a tobacco product selected from the group of smoking tobacco, chewing tobacco as well tobacco substitute products.

In accordance with a particularly preferred embodiment of the invention, a consumer product as defined herein before is provided, wherein said consumer product further comprises at least one, preferably at least two, most preferably at least three other flavour imparting substances or fragrance imparting substances as defined herein before.

In another aspect of the invention the use of a cold receptor stimulant as defined herein before is provided, for imparting and/or enhancing a physiological cooling effect in a consumer product selected from foodstuffs, beverages, oral care products, cosmetic products, personal products and tobacco products.

Specific examples and preferred embodiments of said products are given above. Typically, the use comprises incorporation of the cold receptor stimulant in said products in the amounts recited here above.

In yet another aspect of the invention, a method of imparting or enhancing a physiological cooling effect in a consumer product selected from foodstuffs, beverages, oral care products, cosmetic products, personal care products and tobacco products, is provided, said method comprising incorporating in said product an effective amount of a cold receptor stimulant as defined herein before.

According to a preferred embodiment of the invention, a method as defined herein before is provided, wherein said amount is an amount ranging from 0.0001 to 500 ppm, based on the total weight of said consumer product, preferably an amount ranging from 0.0005 to 50 ppm, more preferably ranging from 0.001 to 10 ppm, most preferably ranging from 0.002 to 5 ppm.

Still another aspect of the invention provides a method of preparing the cold receptor stimulant represented by formula (I) or (II), or an ester or a salt thereof. Typically said process comprises reacting a menthane carboxylic acid with an amine represented by the following formula (III):

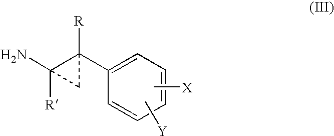

or a salt or ester thereof, wherein R, R′, X, Y and the dashed lines have the same meaning as defined herein above, in relation to formula (I) and (II). These amines are commercially available and/or the person skilled in the art will know how to synthesize them from other commercially available starting materials. The menthane carboxylic acid is preferably an activated menthane carboxylic acid, i.e a menthane carboxylic acid that is more reactive than the original carboxylic acid, as is understood by the person skilled in the art. The term activated carboxylic acid, typically encompasses (mixed) anhydrides, esters, acid chlorides or acid bromides of the carboxylic acid, as will be recognized by the skilled person. In accordance with the present invention the process typically comprises combining the aforementioned starting materials and heating the mixture. The present method of preparing flavour modulating substances comprises processes that make use of conventional reactions. Preferably said reaction is performed by heating the reactants in an organic solvent or a mixture of organic solvents, e.g. DMF/pyridine, for a period of between 0.1-10 hours. According to the present processes the reaction products can typically be isolated by evaporating the solvent and optionally further purified using any of the techniques known by the skilled person, such as chromatography and crystallization.

The present invention as described herein will now be illustrated by means of the following examples, which are in no way intended to limit the scope of protection sought, as defined in the claims.

### EXAMPLES

### Example 1

### Preparation of 2-isopropyl-5-methyl-cyclohexanecarboxylic acid (2-hydroxy-2-phenylethyl)-amide

To a solution of 1.5 g 2-amino-1-phenylethanol in 15 g of methylene chloride was added 2 g of triethylamine at room temperature. To this solution was added gradually at room temperature, without cooling or heating, 2 g of p-menthane-3-carbonyl chloride. During the addition the reaction mixture starts to reflux at 35° C. Stirring was continued for 1 hour at room temperature. The mixture was diluted with 25 g of diethylether and washed twice with 50 ml of a 5% hydrochloric acid solution. After discarding the water layer, the organic layer was washed twice with a saturated sodium bicarbonate solution. Solvent was evaporated and 2 g of the 2-isopropyl-5-methyl-cyclohexanecarboxylic acid (2-hydroxy-2-phenylethyl)-amide was obtained. The molecular structure was confirmed using mass spectroscopy, ¹H NMR and ¹³C NMR.

### Example 2

### Preparation of 2-isopropyl-5-methyl-cyclohexanecarboxylic acid phenylethyl-amide

To a solution of 1.5 g 2-phenylethylamine in 15 g of p-menthane-3-carbonyl chloride 2 g of triethylamine was added at room temperature. To this solution 2 g of menthanoyl chloride was added gradually at room temperature, without cooling or heating. During the addition the reaction mixture starts to reflux at 35° C. Stirring was continued for 1 hour at room temperature. The mixture was diluted with 25 g of diethylether and washed twice with 50 ml of a 5% hydrochloric acid solution. After discarding the water layer, the organic layer was washed twice with a saturated sodium bicarbonate solution. The solvent was evaporated and a very viscous residue was obtained. After cooling to 0° C. the product crystallized. Some pentane was added and the product was filtered. The product was washed once with pentane and dried in a vacuum oven at 60° C./10 mbar. The yield was 1 g of 95% pure 2-isopropyl-5-methyl-cyclohexanecarboxylic acid phenylethyl-amide. The molecular structure was confirmed using mass spectroscopy, ¹H NMR and ¹³C NMR.

### Example 3

### Comparative Sensorial Evaluation of the Product of Example 1

Four aqueous solutions were prepared:

A. 20 ppm menthol;
B. 2 ppm product as obtained in example 1 dissolved in solution A;
C. 20 ppm WS-3 dissolved in solution A; and
D. 2 ppm WS-3 dissolved in solution A.

These solutions were tasted and evaluated by a panel of flavourists. There was general consensus on the results, which were as follows: Solution A was described as: “cooling”; Solution B was described as: “high initial cooling burst”, “long-lasting cooling”, “cold perception in whole mouth”, “some hotness”; Solution C was described as: “more neutral cold perception than B”, “less aggressive cooling than B”, “less long-lasting cooling than B”; Solution D was described as: “more neutral cold perception than B”, “less aggressive cooling than B”, “less long-lasting cooling than B”; Solution B has a comparable strength to solution C and is much stronger than solution D.

### Example 4

### Comparative Sensorial Evaluation of the Product of Example 2

Four aqueous solutions were prepared:

A. 20 ppm menthol;
B. 1 ppm product as obtained in example 2 dissolved in solution A;
C. 20 ppm WS-3 dissolved in solution A; and
D. 2 ppm WS-3 dissolved in solution A.

These solutions were tasted and evaluated by a panel of flavourists. There was general consensus on the results, which were as follows: solution A was described as: “cooling”; solution B was described as: “cold perception with a pain sensation”, “aggressive, long-lasting cooling”, “almost noxious cold”, “slightly tingling”; solution C was described as: “more neutral cold perception than B”, “less aggressive cooling than B”, “less long-lasting cooling than B”; solution D was described as: “more neutral cold perception than B”, “less aggressive cooling than B”, “less long-lasting cooling than B”; solution B has a comparable strength to solution C and is much stronger than solution D.

### Example 5

### Hard Boiled Sweets Containing Cold Receptor Stimulant; Comparative Example

Three different hard boiled sweets were prepared using the formulations as given in table 1.

TABLE I
hard boiled sweets formulation.

| | | | B (Improved | | C (Improved | |
|---|---|---|---|---|---|---|
| Ingredients | A (Control) | | version) | | version) | |
| Sugar | 220 | (g) | 220 | (g) | 220 | (g) |
| Water | 75 | (g) | 75 | (g) | 75 | (g) |
| Glucose syrup 35 DE | 100 | (g) | 100 | (g) | 100 | (g) |
| WS-3 | 50 | (ppm) | | | | |
| Product as prepared | | | 5 | (ppm) | | |
| in example 1 | | | | | | |
| Product as prepared | | | | | 2.5 | (ppm) |
| in example 2 | | | | | | |

The sweets were prepared according to the following procedure. Sugar, water and glucose syrup are mixed and boiled till 146° C. The mix is cooled to 120° C., the cold receptor stimulant is added and carefully mixed in. The mix is poured on a cooling table and folded in and molded on a roller.

The sweets prepared were evaluated by a panel of experienced flavourists. Product A was described as “giving coolness and a cold perception” and “having an immediate cooling effect” and “not longlasting”. Product A containing WS-3 gives at a dosage of 50 ppm WS-3 an irritating feeling in the throat. Product B comprising the product prepared in example 1, was described as “giving a cold sensation which is not irritating” and “much more longlasting than A”. The effect of product B is in the whole mouth and has a tingling-cool aftertaste. Product C comprising the product prepared in example 2, was described as “slow release cooling”, “giving a cold effect in the whole mouth”, “tingling pain sensation enhancing the cooling effect and “very longlasting”. The strength of products A, B and C are comparable.

### Example 5

### Preparation of N-(2-ethoxyphenethyl)-2-isopropyl-5-methylcyclohexane-carboxamide

The title compound was prepared following the general procedure of Example 1.

¹H NMR (MeOD) δ: 7.21-7.13 (m, 2H), 6.94-6.73 (m, 2H), 4.11-3.98 (m, 2H), 3.50-3.32 (m, 2H), 2.87-2.75 (t, 2H), 2.20-2.03 (t. 1H), 1.78-1.49 (m, 4H), 1.46-1.37 (t, 4H), 1.33 (s, 1H), 1.21-0.92 (m, 3H), 0.91-0.82 (t, 6H), 0.77-0.68 (d, 3H).

LC-MS: 332.3 (M⁺). 354.2 (M⁺²³).

### Example 6

### Preparation of 2-isopropyl-5-methyl-N-((1S,2S)-2-phenylcyclopropyl)-cyclohexanecarboxamide

The title compound was prepared following the general procedure of Example 1.

¹H NMR (MeOD) δ: 7.29-7.06 (m, 5H), 2.88-2.78 (m, 1H), 2.23-2.09 (t, 1H), 2.00 (s, 1H), 1.80-1.60 (m, 4H), 1.59-1.46 (t, 1H), 1.38 (s, 1H), 1.26-0.95 9m, 5H), 0.93-0.88 (d, 6H), 0.85-0.76 (d, 3H).

LC-MS: 300.0 (M⁺), 322.2 (M⁺²³).

### Example 7

### Preparation of N-(3,4-dimethylphenethyl)-2-isopropyl-5-ethylcyclo-hexanecarboxamide

The title compound was prepared following the general procedure of Example 1.

¹H NMR (DMSO) δ: 7.91-7.70 (t, 1H), 7.08-6.98 (d, 1H), 7.96-7.84 (m, 2H), 3.32-3.09 (m, 2H), 2.67-2.55 (t, 2H), 2.22-2.10 (d, 6H), 2.08-1.97 (t, 1H), 1.75-1.20 (m, 6H), 1.11-1.00 (m, 1H), 0.93-0.76 (m, 8H), 0.72-0.61 (d, 3H).

LC-MS: 338.3 (M⁺²³).

### Example 8

### Preparation of N-(4-hydroxyphenethyl)-2-isopropyl-5-methylcyclohexanecarboxamide

The title compound was prepared following the general procedure of Example 1.

GC-MS: 303 (M). 288, 184, 139, 120, 107, 83, 69, 55, 41, 30.

### Example 10

### Comparative Sensorial Evaluation of the Products of Examples 5-9

A small group of panelists was asked to taste various aqueous solutions of compounds of formula (I) and indicate which solutions had a cooling intensity similar to or slightly higher than that of a solution of menthol at 2 ppm. The results are shown in Table 1.

TABLE I

| Chemical | Example | Concentration | Odor |
|---|---|---|---|
| Comparison: 1-Menthol | | 2.0 ppm | Minty |
| Comparison: N-ethyl p-menthanecarboxamide (WS-3) | | 1.5 ppm | None |
| N-(2-hydroxy-2-phenylethyl)-2-isopropyl-5- | 1 | 0.005 ppm | None |
| methylcyclohexanecarboxamide. | | | |
| 2-isopropyl-5-methyl-N- | 2 | 0.2 ppm | None |
| phenethylcyclohexanecarboxamide. | | | |
| N-(2-ethoxyphenethyl)-2-isopropyl-5- | 5 | 0.002 ppm | None |
| methylcyclohexanecarboxamide. | | | |
| 2-isopropyl-5-methyl-N-((1S,2S)-2- | 6 | 0.1 ppm | None |
| phcnylcyclopropyl)cyclohexanecarboxamide. | | | |
| N-(3,4-dimethylphenethyl)-2-isopropyl-5- | 7 | 0.002 ppm | None |
| methylcyclohexanecarboxamide. | | | |

1. Cold receptor stimulant selected from the group of substances of the following formula (I): 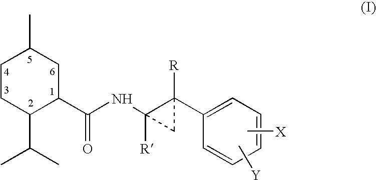
wherein,
R represents hydrogen, hydroxyl, oxo, lower alkyl or lower alkoxyl;
R′ represents hydrogen or lower alkyl;
X and Y independently represent hydrogen, hydroxyl, lower alkyl and lower alkoxyl;
and wherein the dashed lines represent an optional additional bond or an optional C₁-C₂ alkylene moiety; and esters thereof.

2. Cold receptor stimulant according to claim 1, wherein R represents hydrogen, hydroxyl, methyl, ethyl, methoxy, ethoxy, isopropoxy, or propoxy; R′ represents hydrogen, methyl or ethyl; X and Y independently represent hydrogen, methyl, ethyl, propyl, isopropyl, hydroxyl, methoxy, ethoxy, isopropoxy or propoxy and the dashed lines represent an optional double bond or an optional methylene moiety.

3. Cold receptor stimulant according to claim 1, wherein carbon atom no. 1 is in the R-configuration, carbon atom no. 2 is in the S-configuration and wherein carbon atom no. 5 is in the R-configuration.

4. Cold receptor stimulant according to claim 1, selected from the group of 2-isopropyl-5-methyl-cyclohexanecarboxylic acid (2-hydroxy-2-phenylethyl)-amide, 2-isopropyl-5-methyl-cyclohexanecarboxylic acid phenylethyl-amide, N-(2-ethoxyphenethyl)-2-isopropyl-5-methylcyclohexanecarboxamide, 2-isopropyl-5-methyl-N-((1S,2S)-2-phenylcyclopropyl)cyclohexanecarbox-amide, N-(3,4-dimethylphenethyl)-2-isopropyl-5-methylcyclohexanecarboxamide and esters thereof.

5. Cold receptor stimulant according to claim 1, selected from the group of substances of the following formula (II): 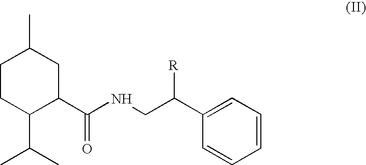
wherein,
R represents hydrogen, hydroxyl, lower alkyl or lower alkoxyl; and esters thereof.

6. Cold receptor stimulant according to claim 5, wherein R represents hydrogen or hydroxyl.

7. Composition comprising a cold receptor stimulant as defined in claim 1 and at least 0.1 wt % of one or more flavour imparting substances or one or more fragrance imparting substances.

8. Composition according to claim 7, comprising at least 2 ppm of the cold receptor stimulant.

9. Composition according to claim 7, wherein the composition comprises the one or more flavour imparting substances or the one or more fragrance imparting substances in an amount within the range of 1-50 wt. % calculated on dry solids weight.

10. Consumer product selected from foodstuffs, beverages, oral care products, cosmetic products, personal care products and tobacco products, comprising a cold receptor stimulant as defined in claim 1, in an amount effective to impart or enhance therein a physiological cooling effect.

11. Consumer product according to claim 10, wherein said amount is an amount ranging from 0.0001 to 500 ppm, based on the total weight of said product.

12. Consumer product according to claim 10, further comprising at least one other flavour imparting substance or at least one other fragrance imparting substance.

13. Consumer product according to claim 10, said product being selected from the group of hard and soft candies, chewing gums, edible films, lozenges, pastilles, desserts, ice creams, soft drinks, alcoholic beverages, dairy drinks, toothpastes, mouthwashes, dental floss, anti-plaque compositions, anti-gingivitis compositions, deodorants, shampoos, skin sanitizing compositions, lotions, shaving products, smoking tobacco, chewing tobacco and tobacco substitute products.

14. (canceled)

15. Method of imparting or enhancing a physiological cooling effect in a consumer product selected from foodstuffs, beverages, oral care products, cosmetic products, personal care products and tobacco products, comprising incorporating in the consumer product an effective amount of a cold receptor stimulant according to claim 1.

16. Method according to claim 15, wherein said effective amount is an amount ranging from 0.0001 to 500 ppm, based on the total weight of said consumer product.

17. Composition according to claim 8, wherein the composition comprises the one or more flavour imparting substances or the one or more fragrance imparting substances in an amount within the range of 1-50% wt. %, calculated on dry solids weight.

18. Consumer product according to claim 11, further comprising at least one other flavour imparting substance or at least one other fragrance imparting substance.

19. Consumer product according to claim 11, said product being selected from the group of hard and soft candies, chewing gums, edible films, lozenges, pastilles, desserts, ice creams, soft drinks, alcoholic beverages, dairy drinks, toothpastes, mouthwashes, dental floss, anti-plaque compositions, anti-gingivitis compositions, deodorants, shampoos, skin sanitizing compositions, lotions, shaving products, smoking tobacco, chewing tobacco and tobacco substitute products.

20. Consumer product according to claim 12, said product being selected from the group of hard and soft candies, chewing gums, edible films, lozenges, pastilles, desserts, ice creams, soft drinks, alcoholic beverages, dairy drinks, toothpastes, mouthwashes, dental floss, anti-plaque compositions, anti-gingivitis compositions, deodorants, shampoos, skin sanitizing compositions, lotions, shaving products, smoking tobacco, chewing tobacco and tobacco substitute products.
